(19) United States
(12) Patent Application Publication
(10) Pub. No.: US 2006/0287660 A1
(43) Pub. Date: Dec. 21, 2006

(54) Electrically Stimulating Nerve Regeneration

(76) Inventors: Naweed I. Syed, Calgary (CA); Graham Arnold Jullien, Calgary (CA)

Correspondence Address: FISH & RICHARDSON P.C., PO BOX 1022, MINNEAPOLIS, MN, 55440-1022, US

(21) Appl. No.: 11/424,413

(22) Filed: Jun. 15, 2006

Related U.S. Application Data
(60) Provisional application No. 60/691,322, filed on Jun. 15, 2005.

(51) Int. Cl. A61B 17/08

(52) U.S. Cl. 606/152

(57) ABSTRACT

This document pertains generally to the field of nerve regeneration and more particularly to functional recovery after nerve injury or transection. For example, this document provides a silicon chip device coupled with field effect (or other types of) transistors, growth permissive chemical substrates, and trophic molecules that together can enable the rapid and successful regeneration of injured nerves. Such devices can be used to stimulate continually the transected target tissue to create an environment that is highly conducive to growth and reinnervation.

### CROSS-REFERENCE TO RELATED APPLICATIONS

This application claims the benefit of U.S. Provisional Application Ser. No. 60/691,322, filed Jun. 15, 2005, the disclosure of which is incorporated by reference in its entirety.

### BACKGROUND

1. Technical Field

This document relates to methods and materials involved in electrically and/or chemically promoting nerve regeneration through a conduit. For example, this document provides silicon chip devices coupled with field effect transistors, growth permissive chemical substrates and trophic molecules that enable the rapid and successful regeneration of injured nerves. The devices can continually stimulate a transected target tissue to maintain its viability during regeneration, thus ultimately creating an environment that is highly conducive to growth and reinnervation.

2. Background Information

Damage to human peripheral nerves is common and debilitating. Despite surgical repair of transected major peripheral nerves, regeneration is frequently incomplete and misdirected, resulting in disability and neuropathic pain. In particular, nerve transection is associated with notoriously poor outgrowth compared to crush or other injuries, especially when the distance between the injury and the target (e.g., muscle or skin) is large. A patient with a brachial plexus injury at the shoulder level is unlikely ever to regain hand function or sensation. Finding ways to improve outgrowth after transection is a clinical challenge that has not yet been solved by better techniques to suture nerves together. Clinical practice currently favors spanning transected nerves with harvested autologous sural nerve grafts, an invasive approach that sacrifices another nerve territory. Newly designed bioartificial grafts are also available.

### SUMMARY

This document relates to methods and materials involved in nerve regeneration. For example, this document provides devices designed to create an electric field (e.g., galvanotroprism) to promote nerve regeneration as well as methods of using such devices to promote nerve regeneration. In some cases, the devices provided herein can have electrodes spaced in small groves (e.g., 10-20 microns) designed for nerve regeneration. For example, a device provided herein can have a tube shape with electrodes spaced along the tube and designed to create electric fields along the length of the tube containing regenerating nerves. Electric fields can be generated throughout the entire cavity in a highly regulated manner or applied exclusively to those portions of the cavity lacking nerve tissue. For example, as regenerating fibers along the cavity make contact with electrodes generating an electric field, the resulting change in capacitive current can provide a signal that the contacted electrodes can be switched off and the neighboring (e.g., next set of electrodes in a series) can be switched on. This can create a gradient of applied electric fields throughout the cavity or tube. In other words, as nerve tissue advances along a cavity, the electrodes in a previously unoccupied segment of the cavity can be switched from an electric field “on state” to an electric field “off state.”

In some cases, the devices provided herein can contain growth permissive chemical substrates (e.g., fibronectin, laminin, etc.), trophic molecules (e.g., nerve growth factor, etc.) or both. For example, the electrodes generating an electric field can be embedded in small cavities containing charged (e.g., positively or negatively charged) quantum dots coated with one or more trophic factors. The quantum dots and their associated trophic factors can then be activated via capacitive current applied through the electrode. In one embodiment, the devices provided herein can have a tube containing a flexible circuit substrate that allows arbitrary voltages to be applied onto an array of conducting tracks (e.g., electrodes) using field effect (or other types of) transistors. Such devices can contain a pump (e.g., a micro-pump) and particles (e.g., nano-beads) that can be activated electrically to help promote nerve fiber growth in a controlled manner.

The methods and materials provided herein can allow for rapid and successful regeneration of injured nerves. For example, the devices provided herein can allow clinicians to treat a mammal (e.g., a human) having an injured nerve.

In general, one aspect of this document features a system that allows for the rapid regeneration of nerves, the system comprising:
a. a tube housing comprising, or formed from, a flexible circuit substrate;
b. one or more integrated circuits, comprising arrays of a definable number of transistors, the circuits bonded to the flexible substrate so as to electrically connect to electrodes on the inside of the tube housing;
c. a microcontroller connected to the integrated transistor array which controls voltages switched onto tracks of the flexible substrate;
d. means for powering the integrated circuits using either wired or wireless connections;
e. an integrated micro-pump;
f. particles that contain at least one trophic factor;
g. an external power source; and
h. an external microcomputer having communication means with the integrated circuit(s).

In some embodiments, the controller may be integrated with the transistor array into the same integrated circuit(s). In some embodiments, the integrated circuit(s) contain array(s) of transistors together with a processor. The electrodes on the circuit substrates can be arranged in a circular manner in the first dimension and, in one embodiment, in the second dimension in an orthogonal and lateral manner on the inside of the tube housing. FIG. 1 shows views of a flexible circuit substrate. A ground plane can shield the connections between the integrated circuit(s) and the electrodes allowing defined electric field profiles to be generated by the electrodes. The tube housing can comprise a flexible multi-sided circuit board manufactured from material that is non-toxic. The tube can be coated with substrate adhesion molecules such as, but not limited to, fibronectin, laminin, and collagen.

The array of transistors can generate a controllable electrical field within the tube housing. The tube also can contain a micro-pump configured to deliver one or more agents (e.g., growth factors) in a controlled and systematic manner. For example, at least one trophic factor can be contained in groves or coated onto particles (e.g., nano-beads). The factors and electrical fields can work together to promote nerve growth and reinnervation.

In some embodiments, the devices provided herein can comprise a wired or wireless connection to the external microcomputer and power source. The external microcomputer can control the activation of the electric field. The power source can provide the power for the electric field.

In general, this document features a system for promoting nerve regeneration. The system comprises, or consists essentially of: (a) a housing comprising a distal end, a proximal end, and a plurality of segments from the distal end to the proximal end, wherein the housing defines a cavity region for nerve regeneration, and wherein each of the plurality of segments comprises an electrode; (b) an integrated circuit configured to control at least one of the electrodes; (c) a pump configured to release an agent into the cavity region; and (d) a power supply configured to provide power to the integrated circuit, the at least one of the electrodes, or the pump. The housing can be tubular. The housing can be tubular with an opening along the length of the housing from the distal end to the proximal end. The width of each of the plurality of segments can be same. Each of the plurality of segments comprises multiple electrodes. The integrated circuit can be attached to the housing. The integrated circuit can be formed within the housing. The system can comprise an integrated circuit for each of the plurality of segments. The housing can be flexible. The pump can define a reservoir comprising the agent. Each of the plurality of segments can comprise at least one of the pumps. Each of the plurality of segments can comprise at least one outlet fluidly connected to the pump. The system can comprise a reservoir configured to be outside a mammal's body, wherein the reservoir is fluidly connected to the pump. The housing can comprise an orthogonal arrangement of electrodes. The housing can comprise at least two orthogonal sets of electrodes on different layers so that each set is electrically isolated from each other. The system can comprise an integrated transistor array. The system can comprise a microcontroller connected to the integrated transistor array. The system can comprises a computer (e.g., a host computer) configured to be outside a mammal's body. A surface of the housing adjacent to the cavity region can be coated with a substrate adhesion molecule. The substrate adhesion molecule can be fibronectin, laminin, or collagen.

In another aspect, this document features a system for nerve regeneration. The system can comprise: (a) a tubular housing comprising a flexible circuit substrate; (b) an arrangement of insulated electrode tracks; (c) one or more integrated circuits for controlling an electrode of the arrangement; (d) a power source to provide the one or more integrated circuits with sufficient voltage to drive an electrode of the arrangement, thereby forming an electric field within the tubular housing; (e) an integrated micro-pump for supplying at least one agent to a region within the tubular housing; (f) an external power source; and (g) an external microcomputer. The one or more integrated circuits can comprise an integrated transistor array. The one or more integrated circuits can comprise a microcontroller. The tubular housing or circuit substrate can comprise a material that is non-toxic. The tubular housing or circuit substrate can be coated with a substrate adhesion molecule. The substrate adhesion molecule can be fibronectin, laminin, or collagen. The integrated circuits can comprise arrays of a definable number of transistors bonded to the flexible substrate so as to electrically connect to an array of insulated electrode tracks formed on the flexible substrate. The integrated circuits can comprise a processor and a voltage driver. An array of transistors inside the integrated circuits drives a set of tracks in a circular arrangement on the inside of the tubular housing and another set of tracks arranged along the length of the tubular housing. The integrated circuits can be capable of generating an electrical field via voltages applied to the arrangement of insulated electrode tracks, wherein the arrangement of insulated electrode tracks are fabricated on the flexible circuit substrate to form, cover, or be on the interior of the tubular housing. The insulated electrode tracks can be on the flexible substrate and are capable of generating electric fields both radially across and along the tubular housing. The micro-pump can be configured to deliver the agent to the region in a controlled and systematic manner. The system can comprise a means for supplying the at least one agent. The means for supplying the at least one agent can comprise nano-beads comprising the at least one agent encapsulated in nano-particles or quantum dots. The means for supplying the at least one agent can release the at least one agent when triggered by an electrical field. The system can be capable of forming an electric field radially across the tubular housing to enable electrophoretic or dielectrophoretic distribution of the at least one agent across a growth cone of a regenerating nerve fiber. The means for supplying the at least one agent can comprise nano-beads. The agent can be a trophic factor. The system can be capable of forming an electric field gradient along the tubular housing to promote growth of a nerve fiber towards a damaged or cut end. The integrated circuits can comprise a wired or wireless connection to the external microcomputer and the external power source. The microcontroller can control the voltages switched onto the integrated electrode tracks of the flexible substrate. The microcontroller can be integrated with a transistor array into at least one of the integrated circuits. The power source can comprise a battery. The power source can be connected to the one or more integrated circuits using a wired or wireless connection. The microcomputer can have communication means with the one or more integrated circuits. The microcomputer and the microcontroller can comprise software and firmware that controls the activation of the integrated transistor array.

In another aspect, this document features a method for promoting nerve regeneration in a mammal having an injured nerve. The method can comprise, or consist essentially of: (a) obtaining a system comprising, or consisting essentially of: (i) a housing comprising a distal end, a proximal end, and a plurality of segments from the distal end to the proximal end, wherein the housing defines a cavity region for nerve regeneration, and wherein each of the plurality of segments comprises an electrode; (ii) an integrated circuit configured to control at least one of the electrodes; (iii) a pump configured to release an agent into the cavity region; and (iv) a power supply configured to provide power to the integrated circuit, the at least one of the electrodes, or the pump; and (b) placing an end of the injured nerve into or proximal to the distal end of the housing. The method can comprise exposing the injured nerve to the agent and electricity from the electrode. The agent can be a nerve growth factor (e.g., NGF).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a top view of one example of a device provided herein having a flexible circuit substrate and attached integrated circuits. In this example, the flexible substrate (e.g., circuit board) can be used as the tube housing. Other examples can have the substrate wrapped around a separate tube.

FIG. 2 is a side view of the device shown in FIG. 1.

FIG. 3 is an end view of the device shown in FIG. 1 with the device being formed in a tube-like configuration. The integrated circuits are in a line so that only the first is visible.

### DETAILED DESCRIPTION

This document provides methods and materials related to nerve regeneration. For example, this document provides devices and methods that can be used to regenerate injured nerves rapidly and successfully. The devices provided herein can provide electric fields (e.g., electrical pulses), chemical agents (e.g., chemical, trophic, and substrate specific molecules), or both to promote nerve regeneration. In one embodiment, a device provided herein can be used to mimic both galvanotropic (e.g., electrical, arrays of controlled field effect (or other types of) transistors and flexible tracks (electrodes)) and chemotrophic (chemical, trophic, and substrate specific molecules) aspects of nervous system development. Such devices can recapitulate aspects of a neurodevelopment program in a microstructure system (e.g., tube housing microstructure system). A device provided herein can allow for the wireless interfacing of nerves with a dimensional array (e.g., a one, two, or three dimensional array) of circularly and laterally arranged tracks (electrodes) driven by an array of field-effect transistors controlled within an integrated circuit. One embodiment includes a method of guiding injured axons toward the source of the applied electrical field while keeping the end-organs “primed” for re-innervation. The devices can comprise a transistor chip that applies a highly controlled, electric field from a flexible substrate that is coated with growth permissive molecules while concurrently delivering the trophic molecules to incoming neurites.

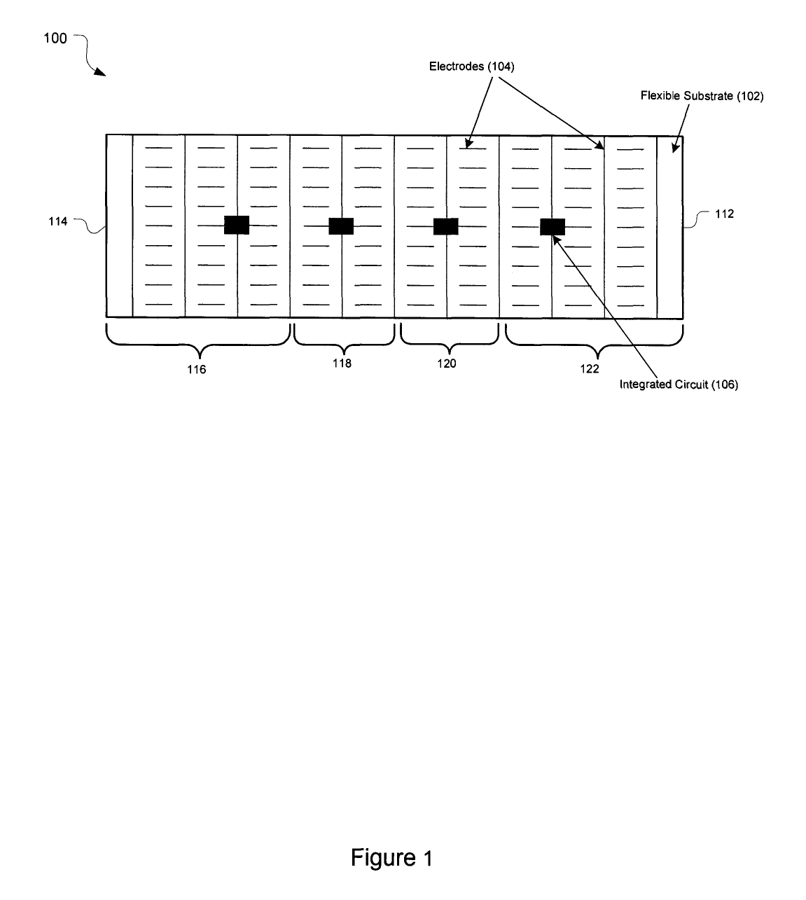

In some cases, a device provided herein can have an array of transistors and a processor in an integrated circuit form (e.g., a transistor chip), which generates an electrical field within a tube (e.g., a tube housing microsystem). Such a tube can have a definable number of tracks on a flexible substrate arranged in a circular and lateral manner and controlled through an array of field effect transistors (or other form of electronic switch) that is connected to a controller (e.g., a microcontroller) which receives commands from an external microcomputer. In the first dimension, rows of tracks (e.g., electrodes) can form a series of rings that line the inside of the tube, and, in the second dimension, orthogonal lateral tracks can form rows along the length of the tube. An example of this arrangement is illustrated in FIG. 1.

A nerve (e.g., a sciatic nerve) be placed in the tube coated with substrate adhesion molecules, such as fibronectin, laminin and collagen. The tube can have an array of ground shielded electrodes, controlled by field effect (or other types of) transistors in an integrated circuit form, which can generate arbitrary dynamic electric fields.

In some cases, a controller (e.g., a microcontroller) can be used to control an integrated pump (e.g., a micro-pump) designed to deliver one or more agents (e.g., trophic factors or growth factors such as nerve growth factor) to the inner cavity of the tube in, for example, a highly controlled and systematic manner. Such agents can be used to form a concentration gradient across the transistor chip. The electrical field can be generated across the first ring of circularly arranged tracks. Together, both the applied electrical field and the release of one or more agents (e.g., trophic factors) can be used to promote growth in an injured nerve.

In some cases, one or more agents such as trophic factors can be delivered directly using, for example, particles such as micro or bio/nano-beads. In one embodiment, a capacitor structure formed on the substrate, when stimulated, can generate an electrical field, which will, in turn, release trophic factor encapsulated in nanoparticles or quantum dots. The electric field can also be controlled to provide a radial electrophoeretic, or dielectrophoretic, force to move the trophic factors away from the sides of the tube and distribute them around the growth cone of the regenerating nerve fiber. In another embodiment, trophic factors can be introduced into the tube and manipulated by electric fields and/or other means such as flow vectors introduced by micro-pumps. The trophic factors can be manipulated to provide a concentration gradient increasing from the growth cone(s) towards an end target (e.g., a fiber end target).

In some cases, a device provided herein can include a means that allows the sensing of the progression of nerve fibers through the tube. In one embodiment, the progression can be monitored using impedance measuring means. As the nerve fibers progress through the tube, the applied field and delivery of trophic factors are controlled by the two-dimensional ring and lateral row structure in the tube such that the field and trophic factors are limited to regions ahead of the nerve fiber ends. Thus, when the fibers reach a specific ring, the next ring of electrodes, ahead of the fiber ends, can be activated which results in the application of an electric field and the release of trophic molecules beyond the fiber ends. The array of electrodes adjacent to the distal end of the nerve can intermittently stimulate the fibers through electric field stimulation. This enables not only the survival of the cut fibers but also prevents the target organ from undergoing tissue dystrophy. This process continues as the nerve fibers grow through the tube housing. These alternating on and off responses in various capacitors and electrodes within the device can cause progressive nerve growth.

The silicon, or other, material used in the conduit can be non-toxic and can enable successful growth of fibers. The cut ends of the nerve can be placed in the device by a surgeon. In one embodiment, a fiber connection (e.g., a nanofiber) can connect the device with an external device that can provide power to the chip and can be affixed externally, adjacent to the site of surgery. In another embodiment, transcutaneous radio frequency (RF) wireless means can be used to both power and provide data to the chip. Most other parameters of the device function are controlled via firmware embedded within the transistor chip and by an external computer (e.g., a microcomputer) communicating with the chip.

The devices provided herein can be configured to provide a conduit whose properties can be altered by local electrical field manipulations from an electronic regeneration interface. Such an interface can have the capacity of fundamentally altering how such nerve injuries are treated by accelerating the rate of growth and manipulation of their molecular properties through rational, sequential time lines with local electronic control circuitry embedded within a transistor chip. The devices also can continually stimulate both the distal and proximal endings, to concurrently manipulate the extracellular and intracellular milieu, thus rendering the microenvironment highly conducive to regeneration and rapid functional recovery.

In one embodiment, a device provided herein can contain electrodes arranged as shown in FIG. 1. For example, device 100 can have a flexible substrate 102 with an array of electrodes 104 on the bottom of flexible substrate 102. Flexible substrate 102 can be made of a polyimide film such as Kapton®. In some cases, flexible substrate 102 can be a multi-layer flexible circuit board material (e.g., Kapton®) with, for example, three layers. Flexible substrate 102 can be a polyimide, and electrodes 104 can be layered on the bottom of flexible substrate 102. In some cases, flexible substrate 102 can be a form of ribbon cable, and electrodes 104 can be formed as an integral component of flexible substrate 102.

Device 100 can contain integrated circuits 106 (four shown in this embodiment). Integrated circuits 106 can be mounted on the top of flexible substrate 102. In some cases, flexible substrate 102 can be used in a roll-to-roll process with amorphous silicon being printed on a flexible plastic substrate with integrated circuits 106 formed as integral components. The number of electrodes are not necessarily the same as shown in FIG. 1. The electrodes can be in any arrangement. Each electrode 104 can be connected to an integrated circuit 106. For example, each electrode 104 can be connected to one of the outputs on an integrated circuit 106.

An integrated circuit 106 can control each electrode within a particular region of device 100. For example, device 100 can have segments 116, 118, 120, and 122, with each segment having one or more electrodes controlled by one or more integrated circuits. Integrated circuits 106 can be custom integrated circuits having voltage driving means to pins on a package from an internal transistor array. The internal transistor array can be driven by a processor (e.g., a microcontroller). The integrated circuits 106 can be interconnected in order to provide a distributed processing environment, or can be driven as peripherals by a separate processor integrated circuit.

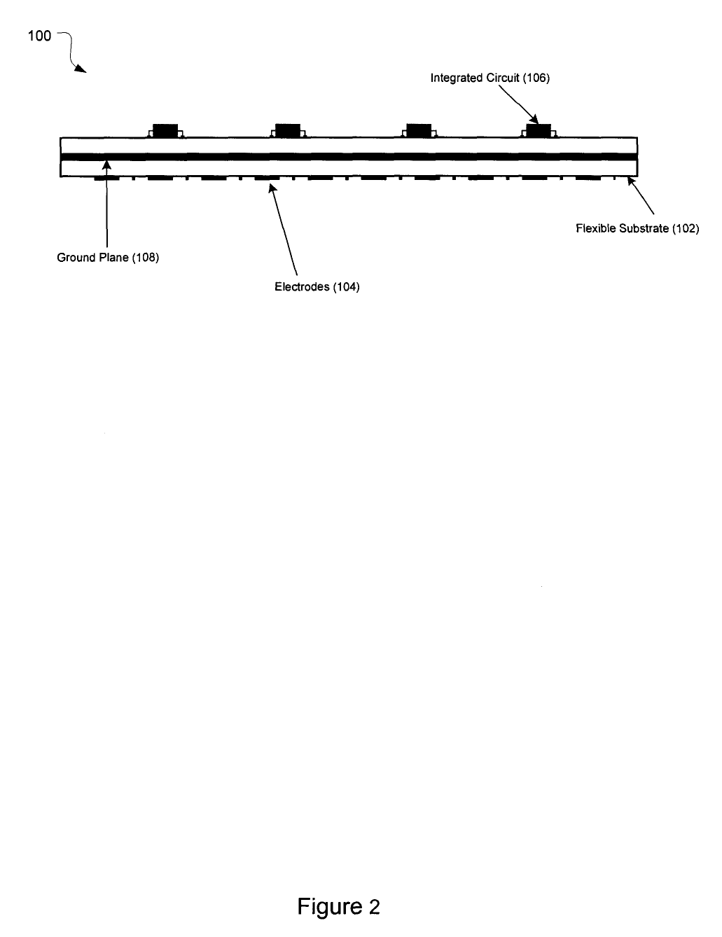

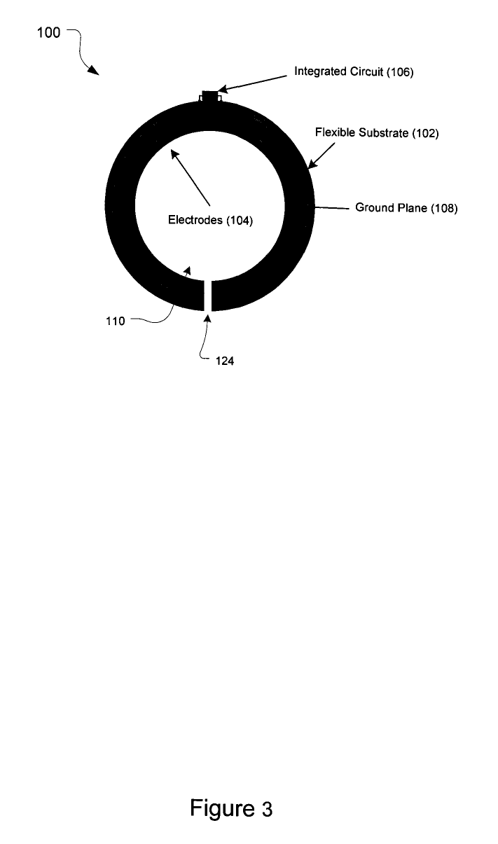

When configured into a tubular structure as shown in FIG. 3, device 100 can define distal end 114 and proximal end 112, which are labeled in FIG. 1. With reference to FIG. 2, device 100 can contain ground plane 108. Ground plane 108 can be a middle layer of a 3-layer flexible circuit substrate connected to a common ground terminal from a power supply. The ground plane can allow accurate control of the electric field below the substrate or within a cavity region without spurious effects from the voltages on interconnecting tracks between the integrated circuit(s) and the electrodes. The ground plane can be patterned to allow interconnections from the upper conducting layer to the lower (electrode) conducting layer without connecting to the ground plane. Device 100 can be configured to have ground plane 108 as the internal layer of a 3-layer multi-layer circuit substrate. The ground plane can be connected to the ground terminal of each integrated circuit.

Device 100 can be configured to have a tubular structure as shown in FIG. 3. Such a tubular structure can define a cavity region 110. Cavity region 110 can provide the space needed for nerve regeneration. The cavity region can be surrounded by flexible substrate 102 or can have one or more openings. For example, device 100 can have a tubular structure with opening 124. Device 100 can be configured such that a cross section of a cavity region has any shape including, a circle (see FIG. 3), oval, square, or rectangle.

In FIG. 3, integrated circuits 106 are in a line so that only the first is visible. Integrated circuits 106 can be in other arrangements such that they do not form a line. For example, integrated circuits 106 can be located at various locations around the outside of a tubular structure.

In some embodiments, a device provided herein can have tracks of passive metal electrodes. A transistor array can be inside one or more integrated circuits and connected to the electrodes using pins on the integrated circuit package. In some cases, the integrated circuits can be directly bonded to the electrodes on a flexible substrate. In these cases, a flexible shield (or extra substrate layer) can be used on top of the bonded chips to protect the bonding.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for promoting nerve regeneration, wherein said system comprises:
(a) a housing comprising a distal end, a proximal end, and a plurality of segments from said distal end to said proximal end, wherein said housing defines a cavity region for nerve regeneration, and wherein each of said plurality of segments comprises an electrode;
(b) an integrated circuit configured to control at least one of said electrodes;
(c) a pump configured to release an agent into said cavity region; and
(d) a power supply configured to provide power to said integrated circuit, said at least one of said electrodes, or said pump.

2. The system of claim 1, wherein said housing is tubular.

3. The system of claim 1, wherein said housing is tubular with an opening along the length of said housing from said distal end to said proximal end.

4. The system of claim 1, wherein the width of each of said plurality of segments is same.

5. The system of claim 1, wherein each of said plurality of segments comprises multiple electrodes.

6. The system of claim 1, wherein said integrated circuit is attached to said housing.

7. The system of claim 1, wherein said integrated circuit is formed within said housing.

8. The system of claim 1, wherein said system comprises an integrated circuit for each of said plurality of segments.

9. The system of claim 1, wherein said housing is flexible.

10. The system of claim 1, wherein said pump defines a reservoir comprising said agent.

11. The system of claim 1, wherein said each of said plurality of segments comprises at least one of said pumps.

12. The system of claim 1, wherein each of said plurality of segments comprises at least one outlet fluidly connected to said pump.

13. The system of claim 1, wherein said system comprises a reservoir configured to be outside a mammal's body, wherein said reservoir is fluidly connected to said pump.

14. The system of claim 1, wherein said housing comprises an orthogonal arrangement of electrodes.

15. The system of claim 1, wherein said housing comprises at least two orthogonal sets of electrodes on different layers so that each set is electrically isolated from each other.

16. The system of claim 1, wherein said system comprises an integrated transistor array.

17. The system of claim 16, wherein said system comprises a microcontroller connected to said integrated transistor array.

18. The system of claim 16, wherein said system comprises a computer configured to be outside a mammal's body.

19. The system of claim 16, wherein a surface of said housing adjacent to said cavity region is coated with a substrate adhesion molecule.

20. The system of claim 16, wherein said substrate adhesion molecule is fibronectin, laminin, or collagen.

21. A system for nerve regeneration, said system comprising:
(a) a tubular housing comprising a flexible circuit substrate;
(b) an arrangement of insulated electrode tracks;
(c) one or more integrated circuits for controlling an electrode of said arrangement;
(d) a power source to provide said one or more integrated circuits with sufficient voltage to drive an electrode of said arrangement, thereby forming an electric field within said tubular housing;
(e) an integrated micro-pump for supplying at least one agent to a region within said tubular housing;
(f) an external power source; and
(g) an external microcomputer.

22. The system of claim 21, wherein said one or more integrated circuits comprises an integrated transistor array.

23. The system of claim 21, wherein said one or more integrated circuits comprises a microcontroller.

24. The system of claim 21, wherein said tubular housing or circuit substrate comprises a material that is non-toxic.

25. The system of claim 21, wherein said tubular housing or circuit substrate is coated with a substrate adhesion molecule.

26. The system of claim 25, wherein said substrate adhesion molecule is fibronectin, laminin, or collagen.

27. The system of claim 21, wherein said integrated circuits comprise arrays of a definable number of transistors bonded to the flexible substrate so as to electrically connect to an array of insulated electrode tracks formed on said flexible substrate.

28. The system of claim 21, wherein said integrated circuits comprise a processor and a voltage driver.

29. The system of claim 21, wherein an array of transistors inside said integrated circuits drives a set of tracks in a circular arrangement on the inside of said tubular housing and another set of tracks arranged along the length of said tubular housing.

30. The system of claim 21, wherein said integrated circuits are capable of generating an electrical field via voltages applied to said arrangement of insulated electrode tracks, wherein said arrangement of insulated electrode tracks are fabricated on said flexible circuit substrate to form, cover, or be on the interior of said tubular housing.

31. The system of claim 21, wherein said insulated electrode tracks are on the flexible substrate and are capable of generating electric fields both radially across and along said tubular housing.

32. The system of claim 21, wherein said micro-pump is configured to deliver said agent to said region in a controlled and systematic manner.

33. The system of claim 21, wherein said system comprises a means for supplying said at least one agent.

34. The system of claim 31, wherein said means for supplying said at least one agent comprises nano-beads comprising said at least one agent encapsulated in nano-particles or quantum dots.

35. The system of claim 31, wherein said means for supplying said at least one agent releases said at least one agent when triggered by an electrical field.

36. The system of claim 33, wherein said system is capable of forming an electric field radially across said tubular housing to enable electrophoretic or dielectrophoretic distribution of said at least one agent across a growth cone of a regenerating nerve fiber.

37. The system of claim 31, wherein said means for supplying said at least one agent comprises nano-beads.

38. The system of claim 31, wherein said agent is a trophic factor.

39. The system of claim 21, wherein said system is capable of forming an electric field gradient along said tubular housing to promote growth of a nerve fiber towards a damaged or cut end.

40. The system of claim 21, wherein said integrated circuits comprise a wired or wireless connection to said external microcomputer and said external power source.

41. The system of claim 23, wherein said microcontroller controls the voltages switched onto said integrated electrode tracks of said flexible substrate.

42. The system of claim 23, wherein said microcontroller is integrated with a transistor array into at least one of said integrated circuits.

43. The system of claim 21, wherein said power source comprises a battery.

44. The system of claim 21, wherein said power source is connected to said one or more integrated circuits using a wired or wireless connection.

45. The system of claim 21, wherein said microcomputer has communication means with said one or more integrated circuits.

46. The system of claim 21, wherein said microcomputer and said microcontroller comprise software and firmware that controls the activation of said integrated transistor array.

47. A method for promoting nerve regeneration in a mammal having an injured nerve, wherein said method comprises:
(a) obtaining a system comprising:
(i) a housing comprising a distal end, a proximal end, and a plurality of segments from said distal end to said proximal end, wherein said housing defines a cavity region for nerve regeneration, and wherein each of said plurality of segments comprises an electrode;
(ii) an integrated circuit configured to control at least one of said electrodes;
(iii) a pump configured to release an agent into said cavity region; and
(iv) a power supply configured to provide power to said integrated circuit, said at least one of said electrodes, or said pump; and
(b) placing an end of said injured nerve into or proximal to said distal end of said housing.

48. The method of claim 47, wherein said method comprises exposing said injured nerve to said agent and electricity from said electrode.

49. The method of claim 47, wherein said agent is a nerve growth factor.
